(19) United States
(12) Patent Application Publication
(10) Pub. No.: US 2026/0315038 A1
(43) Pub. Date: Oct. 15, 2026

(54) METHOD AND SYSTEM FOR PERFORMING ULTRASOUND IMAGING USING A TWO-DIMENSIONAL PROBE

(71) Applicant: GE Precision Healthcare LLC, Waukesha, WI (US)

(72) Inventors: Ke Tao, Wuxi (CN); Minyu Gu, Wuxi (CN); Xingti Liu, Wuxi (CN)

(21) Appl. No.: 19/642,439

(22) Filed: Apr. 8, 2026

(30) Foreign Application Priority Data
Apr. 9, 2025 (CN) 202510443074.0

(51) Int. Cl. A61B 8/00; A61B 8/08

(52) U.S. Cl. CPC A61B 8/5223; A61B 8/0833; A61B 8/4245; A61B 8/461; A61B 8/5207

(57) ABSTRACT

A method for performing ultrasound imaging by using a two-dimensional probe Including: obtaining a two-dimensional ultrasound image of a tissue to be imaged; performing identification on the ultrasound image, and determining a high-acoustic-impedance object in the ultrasound image based on an identification result; continuing to perform ultrasound imaging on the high-acoustic-impedance object by using at least one row of elements among the plurality of rows of elements to obtain a first image of the high-acoustic-impedance object; performing ultrasound imaging on at least a part of an area below the high-acoustic-impedance object by using at least one other row of elements among the plurality of rows of elements to obtain a second image of the at least a part of the area below; and synthesizing the first image and the second image to obtain a real-time synthesized image.

### CROSS-REFERENCE TO RELATED APPLICATION

This application claims priority to Chinese Patent Application No. 202510443074.0, which was file on Apr. 9, 2025 at the Chinese Patent Office. The entire contents of the above-listed application are incorporated by reference herein in their entirety.

### TECHNICAL FIELD

The present application relates to the field of ultrasound imaging, and more specifically, to a method and a system for performing ultrasound imaging by using a two-dimensional probe.

### BACKGROUND

In ultrasound imaging technology, an ultrasound signal is usually transmitted to an object to be imaged by an ultrasound probe, and an image of the object to be imaged is generated by receiving and processing an echo signal. Two-dimensional ultrasound imaging technology is one type of ultrasound imaging technology, and usually uses a two-dimensional probe to perform ultrasound imaging. Specifically, the two-dimensional probe can transmit and receive ultrasound signals in a plane, and the signals in the plane are processed, so that a two-dimensional ultrasound image can be obtained.

A high-acoustic-impedance object in the object to be imaged may affect ultrasound imaging. The high-acoustic-impedance object is an object that causes relatively high acoustic impedance to propagation of a sound wave. Some high-density strong reflectors are common high-acoustic-impedance objects, including interventional needles in puncture procedures, bones, stones, etc. When the sound wave reaches the high-acoustic-impedance object, strong reflection occurs, which causes generation of acoustic shadows, resulting in a reduction in the imaging quality of an area below the high-acoustic-impedance object. For example, in an interventional procedure, generation of acoustic shadows causes a reduction in the image quality of an area below an interventional needle, resulting in a loss of information about the area below the interventional needle and affecting the diagnostic confidence of doctors.

### SUMMARY

Some examples of the present application provide a method for performing ultrasound imaging by using a two-dimensional probe. The two-dimensional probe comprises a plurality of rows of elements. The method comprises: obtaining a two-dimensional ultrasound image of a tissue to be imaged; performing identification on the ultrasound image, and determining a high-acoustic-impedance object in the ultrasound image based on an identification result; continuing to perform ultrasound imaging on the high-acoustic-impedance object by using at least one row of elements among the plurality of rows of elements to obtain a first image of the high-acoustic-impedance object; performing ultrasound imaging on at least a part of an area below the high-acoustic-impedance object by using at least one other row of elements among the plurality of rows of elements to obtain a second image of the at least a part of the area below; and synthesizing the first image and the second image to obtain a real-time synthesized image.

Optionally, the high-acoustic-impedance object comprises at least one of an interventional needle, a bone, a stone, and a calcification.

Optionally, continuing to perform ultrasound imaging on the high-acoustic-impedance object by using at least one row of elements among the plurality of rows of elements comprises: adjusting, based on relative positions of the high-acoustic-impedance object and the at least one row of elements, an ultrasound wave transmission direction of the at least one row of elements to be perpendicular to a surface of the high-acoustic-impedance object.

Optionally, the ultrasound wave transmission method of the at least one row of elements is adjusted in one imaging plane, and the imaging plane passes through the high-acoustic-impedance object.

Optionally, performing ultrasound imaging on the at least a part of the area below by using the at least one other row of elements comprises: adjusting, based on relative positions of the high-acoustic-impedance object and the at least one other row of elements, an ultrasound wave transmission direction of the at least one other row of elements to at least partially pass through the tissue to be imaged below the high-acoustic-impedance object.

Optionally, the ultrasound wave transmission direction of the at least one other row of elements is adjusted in an imaging plane different from that of the at least one row of elements.

Optionally, the method further comprises: determining a key anatomical feature below the high-acoustic-impedance object based on the identification result, wherein the key anatomical feature comprises at least one of a gland, a nerve node, and a blood vessel.

Optionally, performing ultrasound imaging on the at least a part of the area below by using the at least one other row of elements comprises: controlling, based on the position of the key anatomical feature, an ultrasound wave transmission direction of the at least one other row of elements to at least partially pass through the key anatomical feature.

Optionally, the method further comprises: displaying the synthesized image in real time; and highlighting the key anatomical feature in the synthesized image.

Optionally, the plurality of rows of elements comprises three rows of elements, the at least one row of elements comprises a middle row of elements among the three rows of elements, and the at least one other row of elements comprises elements on both sides among the three rows of elements.

Optionally, synthesizing the first image and the second image comprises: selecting a first portion of the first image that comprises the high-acoustic-impedance object and an area above the high-acoustic-impedance object; selecting a second portion of the second image that comprises the area below the high-acoustic-impedance object; and synthesizing the first portion and the second portion.

Optionally, the high-acoustic-impedance object comprises an interventional needle, and the method further comprises: adjusting ultrasound wave transmission directions of the at least one row of elements and the at least one other row of elements in real time in response to a change in the position of the interventional needle in the tissue to be imaged, to continuously obtain an image of the interventional needle and an image of at least a part of an area below the interventional needle.

Some embodiments of the present application further provide an ultrasound imaging system, comprising: a two-dimensional probe; and a processor. The processor is configured to perform any one of the above methods.

Some examples of the present application further provide a non-transitory computer-readable medium. The non-transitory computer-readable medium stores a computer program, the computer program has at least one code segment, and the at least one code segment is executable by a machine to cause the machine to perform the steps of any one of the above methods.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic block diagram of an example of an ultrasound imaging system according to an example of the present application;

FIG. 2 shows a method for performing ultrasound imaging by using a two-dimensional probe according to an example of the present application;

FIG. 3 is a schematic diagram of a two-dimensional probe including a plurality of rows of elements according to an example of the present application;

FIG. 4 is a schematic diagram of a two-dimensional ultrasound image including an interventional needle according to an example of the present application;

FIG. 5 is a schematic diagram of performing two-dimensional ultrasound imaging by using different rows of elements according to an example of the present application; and

FIG. 6 is a schematic diagram of imaging a key anatomical feature according to an example of the present application.

### DETAILED DESCRIPTION

Specific embodiments of the present application are described below. It should be noted that in the specific description of these embodiments, for a concise description, the present application may not describe in detail all of the features of the actual embodiments. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and extensive, for a person of ordinary skill in the art related to the disclosure of the present application, some design, manufacture, or production changes made on the basis of the technical disclosure of the present disclosure are only conventional technical means, and should not be construed as the content of the present disclosure being insufficient.

Unless otherwise defined, the technical or scientific terms used in the claims and the description should be as they are usually understood by those possessing ordinary skill in the technical field to which they belong. The terms “first”, “second”, and similar words used in the present application and the claims do not express any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

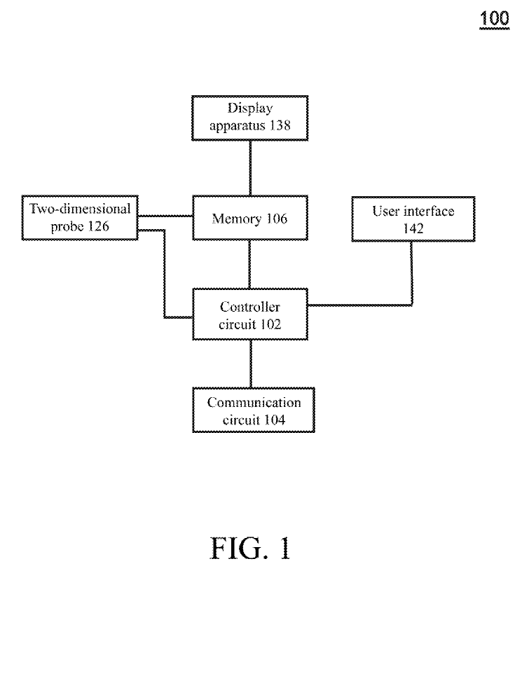

FIG. 1 is a schematic block diagram of an example of an ultrasound imaging system 100. The ultrasound imaging system 100 may include a controller circuit 102 that is operatively connected to a communication circuit 104, a display apparatus 138, a user interface 142, a two-dimensional probe 126, and a memory 106.

The controller circuit 102 is configured to control an operation of the ultrasound imaging system 100. The controller circuit 102 may include one or more processors. Optionally, the controller circuit 102 may include a central processing unit (CPU), one or more microprocessors, a graphics processing unit (GPU), or any other electronic component capable of processing inputted data according to a specific logic instruction. Optionally, the controller circuit 102 may include and/or represent one or more hardware circuits or circuit systems, and the hardware circuit or circuit system includes, is connected to, or includes and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more alternate ultrasound imaging systems, remote servers, etc. The remote server may represent patient information, a machine learning algorithm, a remotely stored medical image from a previous scan, and/or a diagnosis and treatment period of a patient, etc. The communication circuit 104 may represent hardware for transmitting and/or receiving data along a bidirectional communication link. The communication circuit 104 may include a transceiver, a receiver, etc., and an associated circuit system (e.g., an antenna) for communicating (e.g., transmitting and/or receiving) with the one or more alternate ultrasound imaging systems, remote servers, etc., by using a wired and/or wireless means. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternative ultrasound imaging systems, remote servers, etc. The bidirectional communication link may be based on a standard communication protocol, such as Ethernet, TCP/IP, Wi-Fi, 802.11, a customized communication protocol, Bluetooth, etc.

The controller circuit 102 is operatively connected to the display apparatus 138 and the user interface 142. The display apparatus 138 may include one or more liquid crystal display apparatuses (e.g., light emitting diode (LED) backlights), organic light emitting diode (OLED) display apparatuses, plasma display apparatuses, CRT display apparatuses, and the like. The display apparatus 138 may display patient information, one or more medical images and/or videos, a graphical user interface, or a component received by the display apparatus 138 from the controller circuit 102, one or more 2D, 3D, or 4D ultrasound image data sets from ultrasound data stored in the memory 106, or anatomical measurement, diagnosis, and processing information, and the like currently acquired in real time.

The user interface 142 controls the controller circuit 102 and the operation of the ultrasound imaging system 100. The user interface 142 is configured to receive an input from a clinician and/or an operator of the ultrasound imaging system 100. The user interface 142 may include a keyboard, a mouse, a trackball, a touch pad, one or more physical buttons, and the like. Optionally, the display apparatus 138 may be a touch screen display apparatus that includes at least a part of the user interface 142. For example, a part of the user interface 142 may correspond to a graphical user interface (GUI) that is generated by the controller circuit 102 and is shown on the display apparatus 138. The touch screen display apparatus may detect the presence of a touch from the operator on the display apparatus 138, and may also identify the location of the touch relative to the surface area of the display apparatus 138. For example, a user may select, by touching or contacting the display apparatus 138, one or more user interface components of the user interface (GUI) shown on the display apparatus. User interface components may correspond to icons, text boxes, menu bars, etc., shown on the display apparatus 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include one or more learning algorithms (e.g., deep learning algorithms including a convolutional neural network algorithm, machine learning algorithms such as a decision tree learning algorithm, a conventional computer vision algorithm, or the like) configured to define an image analysis algorithm. During execution of the image analysis algorithm, the controller circuit 102 is configured to identify some anatomical features of a medical image. A type of a specific anatomical feature may be selected based on an object described in any one of the following examples of the present application. In some examples, an anatomical feature that can be identified is an object with high acoustic impedance. The high-acoustic-impedance object may be determined based on general understanding in the field of ultrasound imaging, and may be understood as an object having impedance to an ultrasound signal sufficient to affect the imaging quality of an area below. Such high impedance may be caused by strong reflection, absorption, etc., of a sound wave. In different imaging scenarios, a tissue to be imaged may include different high-acoustic-impedance objects. For example, in an ultrasound imaging-assisted puncture procedure, the high-acoustic-impedance object may include an interventional needle. In addition, some physiological structures inside the tissue to be imaged may also become high-acoustic-impedance objects. For example, bones, stones, or calcifications may be understood as high-acoustic-impedance objects. In addition to the high-acoustic-impedance object, the image analysis algorithm in this example of the present application may also be used to identify another anatomical feature. For example, the feature may be a key anatomical feature of the tissue to be imaged, for example, may be at least one of a nodule, a gland, a nerve node, a blood vessel, etc. An example description of a specific configuration manner of the image analysis algorithm is described below.

The image analysis algorithm may be defined by one or more algorithms to identify a section of a subject to be scanned of interest based on one or more anatomical features (e.g., a boundary, thickness, pixel value change, valve, cavity, chamber, edge or inner layer, vessel structure, etc.) within the medical image, a modality or pattern of the medical image (e.g., color blood flow), etc. The one or more anatomical features may represent a feature of pixels and/or voxels of the medical image, such as a histogram of oriented gradients, a point feature, a covariance feature, a binary mode feature, and the like. For example, the image analysis algorithm may be defined by using prediction of object identification within the medical image by using one or more deep neural networks.

The image analysis algorithm may correspond to an artificial neural network formed by the controller circuit 102 and/or the remote server. The image analysis algorithm may be divided into two or more layers, such as an input layer for receiving an input image, an output layer for outputting an output image, and/or one or more intermediate layers. Layers of a neural network represent different groups or sets of artificial neurons, and may represent different functions that are executed by the controller circuit 102 on an input image (e.g., an ultrasound image obtained and/or generated by the ultrasound imaging system 100) to identify the input image. An artificial neuron in a layer of the neural network may examine an individual pixel in the input image. The artificial neurons use different weights in a function applied to the input image, so as to attempt to identify an object in the input image. The neural network produces an output image by assigning or associating different pixels in the output image to or with different anatomical features on the basis of the analysis of pixel characteristics. The output image may include an identification result for the anatomical feature. It may be understood that the identification result may include an identification result of the type of the anatomical feature, e.g., a high-acoustic-impedance object (e.g., at least one of an interventional needle, a bone, a stone, or a calcification), and for another example, a key anatomical feature (e.g., at least one of a nodule, a gland, a nerve node, or a blood vessel). The identification result may further include information such as the position and the size of the feature. For example, the identified anatomical feature may be highlighted in a specific display manner.

With continued reference to FIG. 1, the ultrasound imaging system 100 may include the two-dimensional (2D) probe 126. The two-dimensional probe 126 has elements (not shown) such as an ultrasound transducer, a transmitter, a transmit beamformer, and a detector/SAP electronics. The detector/SAP electronics may be used to control switching of transducer elements. The detector/SAP electronics may also be used to group the transducer elements into one or more sub-holes. The two-dimensional probe 126 may be any type of probe, including a linear probe, a curved array probe, a 1.25D array probe, a 1.5D array probe, or a 1.75D array probe. Compared with another probe such as a four-dimensional (4D) probe, the two-dimensional probe 126 can image a two-dimensional plane at a high frame rate while maintaining low power of the probe to mitigate the problem of heating. The two-dimensional probe 126 further has a small volume and is low in cost. The two-dimensional probe 126 may include a plurality of rows of elements (FIG. 1) not shown. For example, the two-dimensional probe 126 may include three rows of elements, or may include five or more rows of elements.

The two-dimensional probe 126 may be configured to obtain ultrasound data or information from a tissue to be imaged (e.g., organs such as a breast, a heart, a prostate, and corresponding skin surfaces outside the organs) of a patient. The two-dimensional probe 126 is communicatively connected to the controller circuit through the transmitter. The transmitter transmits a signal to the transmit beamformer on the basis of acquisition settings received by the controller circuit 102. The acquisition settings may define the amplitude, pulse width, frequency, gain setting, scanning angle, power, time gain compensation (TGC), resolution, and the like of the ultrasound pulses transmitted by the ultrasound transducer. The ultrasound transducer transmits a pulsed ultrasound signal into a patient (e.g., the body). The acquisition settings may be defined by a user operating the user interface 142. The signal transmitted by the transmitter, in turn, drives the ultrasound transducer.

The ultrasound transducer transmits the pulsed ultrasound signal to a body (e.g., a patient) that corresponds to an acquisition setting along one or more scanning planes. The ultrasound signal may include, for example, one or more reference pulses, one or more push pulses (e.g., shear waves), and/or one or more pulsed wave Doppler pulses. At least a portion of the pulsed ultrasound signal is backscattered from a tissue to be imaged (e.g., an organ, bone, heart, breast tissue, liver tissue, cardiac tissue, prostate tissue, newborn brain, embryo, abdomen, etc.) to produce an echo. Depending on the depth or movement, the echo is delayed in time and/or frequency, and received by the ultrasound transducer. The ultrasound signal may be used for imaging, for producing and/or tracking a shear wave, for measuring changes in position or velocity within an anatomical structure and a compressive displacement difference (e.g., strain) of a tissue, and/or for treatment and other applications. For example, the two-dimensional probe 126 may deliver low energy pulses during imaging and tracking, deliver medium and high energy pulses to produce shear waves, and deliver high energy pulses during treatment.

The ultrasound transducer converts received echo signals into electrical signals that can be received by the receiver. The receiver may include one or more amplifiers, analog/digital converters (ADCs), and the like. The receiver may be configured to amplify the received echo signals after appropriate gain compensation, and convert these analog signals received from each transducer element into digitized signals that are temporally uniformly sampled. The digitized signals representing the received echoes are temporarily stored in the memory 106. The digitized signals correspond to the backscattered waves received by each transducer element at different times. After being digitized, each signal may still retain the amplitude, frequency, and phase information of the backscattered wave.

Optionally, the controller circuit 102 may retrieve the digitized signals stored in the memory 106 for use in a beam forming processor. For example, the controller circuit 102 may convert each digitized signal into a baseband signal or compress the digitized signal.

In some embodiments, the controller circuit 102 may further include a beam forming processor. The beam forming processor may include one or more processors. If desired, the beam forming processor may include a central processing unit (CPU), one or more microprocessors, or any other electronic component capable of processing the input data according to specific logic instructions. Additionally or alternatively, the beam forming processor may execute instructions stored on a tangible and non-transitory computer-readable medium (e.g., the memory 106) to perform beam forming computation using any suitable beam forming method, such as adaptive beam forming, synthetic emission focusing, aberration correction, synthetic aperture, clutter suppression, and/or adaptive noise control, etc.

In some embodiments, the controller circuit 102 may further include a radio frequency (RF) processor. The beam forming processor executes beam forming on the digitized signals of the transducer elements, and outputs an RF signal. The RF signal is then provided to the RF processor for processing the RF signal. The RF processor may include one or more processors. If desired, the RF processor may include a central processing unit (CPU), one or more microprocessors, or any other electronic component capable of processing the inputted data according to specific logic instructions. Additionally or alternatively, the RF processor may execute instructions stored on a tangible and non-transitory computer-readable medium (e.g., the memory 106). Optionally, the RF processor may be integrated with and/or be part of the controller circuit 102. For example, operations described as being executed by the RF processor may be configured to be executed by the controller circuit 102.

The RF processor may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor may generate tissue Doppler data for multiple scanning planes. The RF processor acquires the information (e.g., I/Q, B-mode, color Doppler, tissue Doppler, and Doppler energy information) related to a plurality of data pieces, and stores the data information in the memory 106, where the data information may include time stamp and orientation/rotation information.

Optionally, the RF processor may include a composite demodulator (not shown) for demodulating the RF signal to generate an IQ data pair representing an echo signal. The RF or IQ signal data may be provided directly to the memory 106 so as to be stored (e.g., stored temporarily). If desired, an output of the beam forming processor may be delivered directly to the controller circuit 102.

The controller circuit 102 may be configured to process the acquired ultrasound data (e.g., RF signal data or IQ data pairs), and prepare and/or generate frames of ultrasound image data representing an anatomical structure of interest so as to display the same on the display apparatus 138. The acquired ultrasound data may be processed by the controller circuit 102 in real time during a scanning or treatment process of ultrasound examination when echo signals are received. Additionally or alternatively, the ultrasound data may be temporarily stored in the memory 106 during a scanning process, and processed in a less real-time manner in a live or off-line operation.

In some examples, the ultrasound imaging system 100 described above may be used to perform two-dimensional ultrasound imaging. An obtained two-dimensional image can be used by a doctor for diagnosis or treatment. In some scenarios, there may be objects with high acoustic impedance inside the tissue to be imaged. This will cause generation of acoustic shadows and affect the quality of ultrasound imaging. At least based on this, improvements are provided in some examples of the present application.

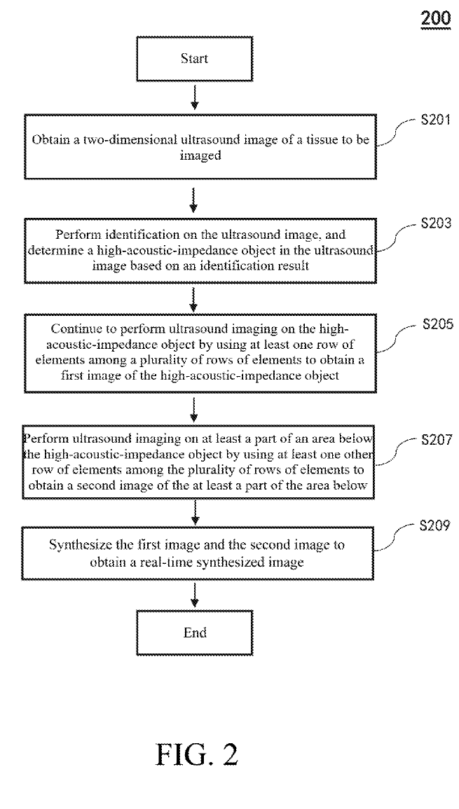

Referring to FIG. 2, FIG. 2 shows a method 200 for performing ultrasound imaging by using a two-dimensional probe according to some examples of the present application. The two-dimensional probe may be the probe including a plurality of rows of elements described in any one of the above examples of the present application. The method may include the following steps.

In step S201, a two-dimensional ultrasound image of a tissue to be imaged is obtained. This process may be implemented by a processor. For example, the processor may control the elements of the two-dimensional probe to transmit ultrasound signals to the inside of the tissue to be imaged and receive echo signals. Further, the processor may process the echo signals to generate the two-dimensional ultrasound image of the tissue to be imaged.

In step S203, identification is performed on the ultrasound image, and a high-acoustic-impedance object in the ultrasound image is determined based on an identification result. This step is performed by the processor. For example, the processor may perform the image analysis algorithm (e.g., an algorithm based on an artificial neural network, or on the basis of another image analysis algorithm) described in any one of the above examples of the present application to perform image identification and determine the high-acoustic-impedance object.

In step S205, ultrasound imaging continues to be performed on the high-acoustic-impedance object by using at least one row of elements among the plurality of rows of elements to obtain a first image of the high-acoustic-impedance object. As described above in the present application, the two-dimensional probe includes a plurality of rows of elements. After determining the high-acoustic-impedance object, the processor may control some rows of elements among the plurality of rows of elements to continue to image the high-acoustic-impedance object. Continuing to image the high-acoustic-impedance object may be understood as controlling at least one row of elements to transmit ultrasound signals toward the high-acoustic-impedance object, so that an imaging plane of the at least one row of elements passes through the high-acoustic-impedance object.

In step S207, ultrasound imaging is performed on at least a part of an area below the high-acoustic-impedance object by using at least one other row of elements among the plurality of rows of elements to obtain a second image of the at least a part of the area below. Thus, the processor can simultaneously control the two-dimensional probe to image an acoustic shadow area below the high-acoustic-impedance object, and the second image can effectively compensate for the insufficient image quality of the acoustic shadow area in the first image.

In step S209, the first image and the second image are synthesized to obtain a real-time synthesized image. After generating the first and second images, the processor may perform synthesis processing on the first and second images to obtain a complete two-dimensional image of the tissue to be imaged. The above process is performed in real time, and thus, when performing ultrasound scanning by using the above embodiment of the present application, a doctor can obtain a high-quality ultrasound image in real time. It should be noted that “real-time” allows for short delays, such as delays in transmission and reception of ultrasound signals, and small delays in an image processing process.

In the above embodiment of the present application, the plurality of rows of elements originally used for two-dimensional ultrasound imaging in the same plane are respectively applied to imaging of the plane in which the high-acoustic-impedance object is located and imaging of the acoustic shadow area caused by the acoustic impedance object, thereby effectively eliminating the acoustic shadow area and improving the image quality.

Such a manner can be used in a variety of different application scenarios to improve the imaging quality. For example, when lesion screening is performed, an acoustic shadow caused by a high-acoustic-impedance object such as a calcification or a stone will affect the image quality below the high-acoustic-impedance object. Based on the method described in the above example of the present application, the quality of a two-dimensional image can be effectively improved and an acoustic shadow can be eliminated in a real-time imaging process, thereby avoiding situations in which structures such as fine lesions in an area below cannot be effectively observed due to the reduced image quality. For another example, when a puncture procedure is performed, a puncture needle is used as a high-acoustic-impedance object, and an acoustic shadow area may be formed below the puncture needle. The implementation of the present application can effectively eliminate an acoustic shadow, thereby avoiding a decrease in the surgical confidence caused by the inability of a doctor to observe an entire image during the puncture process.

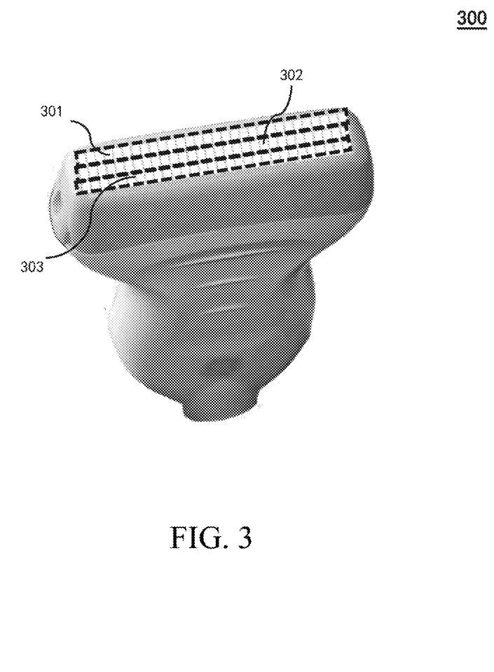

Embodiments of the present application are described in more detail with reference to FIG. 3. FIG. 3 is a schematic diagram of a two-dimensional probe 300 including a plurality of rows of elements. The two-dimensional probe 300 includes three rows of elements 301 to 303. The first row of elements 301 and the third row of elements 303 are located on both sides, and the second row of elements 302 is located in the middle. In a conventional two-dimensional ultrasound imaging mode, the three rows of elements of the two-dimensional probe 300 perform imaging substantially in the same plane. When there is a high-acoustic-impedance object in the imaging plane, downward propagation of an ultrasound wave may be blocked. As a result, an acoustic signal related to a blocked area in the plane cannot be obtained, resulting in formation of an acoustic shadow. In some examples of the present application, at least one row of elements (e.g., the middle row of elements, that is, the second row of elements 302) of the two-dimensional probe 300 may be configured to continue to image the high-acoustic-impedance object. At least one other row of elements (e.g., elements on both sides, that is, the first row of elements 301 and the third row of elements 303) of the two-dimensional probe 300 may be adjusted in terms of ultrasound signal transmission angle, so that an ultrasound signal avoids the high-acoustic-impedance object and passes below the high-acoustic-impedance object. Thus, an acoustic shadow area can be eliminated, and the ultrasound imaging quality can be improved.

It should be noted that FIG. 3 is only an example illustration of the two-dimensional probe. In another example, another quantity of rows of elements may be included in the two-dimensional probe, and may be, for example, more than three rows. In addition, a quantity of elements per row may also be adjusted.

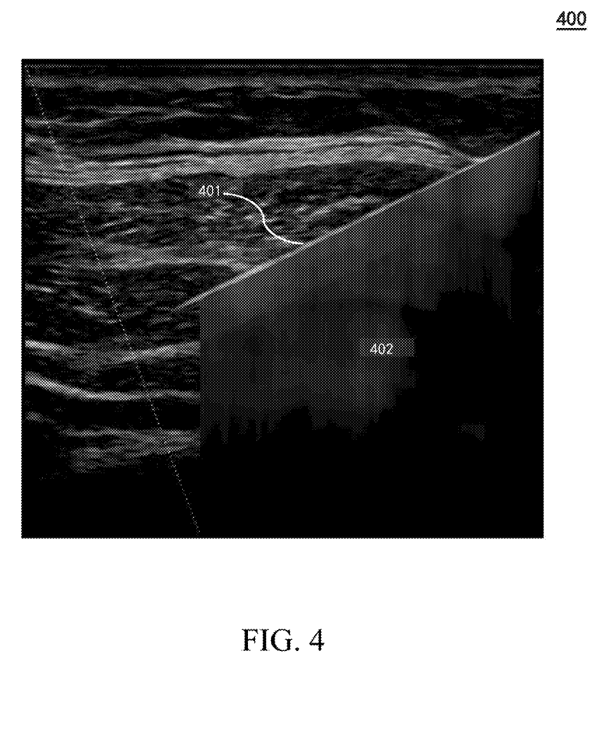

As described above in the present application, the imaging method in the present application is applicable to a scenario in which a plurality of different high-acoustic-impedance objects exist. Description is provided below by using an interventional needle as an example. The interventional needle is usually a needle-shaped object made of a metal material, and is used for lesion sampling, drug administration, etc. When performing an interventional procedure, a doctor often uses two-dimensional ultrasound imaging technology to perform auxiliary imaging, to determine the position of the interventional needle and a distance between the interventional needle and a lesion in real time, and also determine whether there is a sufficient safety distance between the interventional needle and a key anatomical feature. The interventional needle is a type of high-acoustic-impedance object, and the interventional needle may cause generation of an acoustic shadow during a two-dimensional ultrasound imaging process. Detailed description is provided with reference to FIG. 4. FIG. 4 is a schematic diagram of a two-dimensional ultrasound image 400 including an interventional needle 401. The interventional needle 401 appears with high brightness in the image due to a strong echo signal of the needle. In addition, because the interventional needle 401 blocks downward propagation of an ultrasound signal, the image quality is reduced and a shadow appears in an area below the interventional needle 401. As shown in FIG. 4, the area is represented by an acoustic shadow area 402. The acoustic shadow area 402 may cause loss of key information. For example, if key anatomical features such as nodules, nerve nodes, blood vessels, or glands are included in the acoustic shadow area, the doctor needs to clearly know relative positions of the interventional needle 401 and these key features to perform accurate interventional path planning. The appearance of the acoustic shadow area 402 may affect such determining and planning.

A plurality of examples of the present application are developed to address the above issues. For example, the example corresponding to FIG. 2 can eliminate the acoustic shadow area 402 while maintaining two-dimensional ultrasound imaging. Moreover, the examples of the present application do not require the assistance of another probe or replacement of another probe, which can ensure that the imaging process is consistent with original use habits of the doctor.

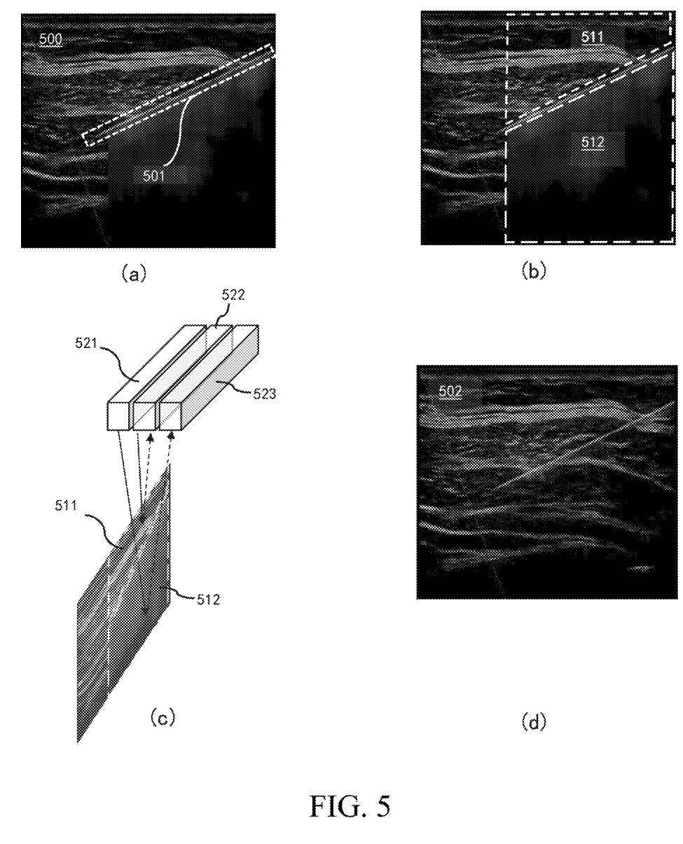

Further referring to FIG. 5, FIG. 5 is a schematic diagram of performing two-dimensional ultrasound imaging by using different rows of elements according to an example of the present application.

FIG. 5a shows image identification of an ultrasound image 500 to determine a high-acoustic-impedance object (taking an interventional needle 501 as an example) in the ultrasound image 500. Due to the presence of the interventional needle 501, the ultrasound image 500 has an acoustic shadow below the interventional needle 501, which causes deterioration of the image quality. Further, for a manner of image identification, refer to that described in any one of the above examples of the present application. For example, image identification may be performed on the ultrasound image 500 by using an artificial neural network to directly determine the interventional needle 501 in the ultrasound image 500. Alternatively, any other type of algorithm may be used, for example, image segmentation is performed by analyzing a grayscale value of the image and setting a specific threshold, and pixel points with grayscale values greater than the specific threshold are obtained through segmentation, to automatically identify the interventional needle 501. The present application does not make a limitation.

Further, as shown in FIG. 5b, after the interventional needle 501 is identified, an area 511 above and an area 512 below the interventional needle 501 may be determined. It should be noted that, to better illustrate the solution of the present application, the area 511 above and the area 512 below are shown with different styles of outlines, which do not represent actual images. The area 511 above is not affected by acoustic impedance of the interventional needle 501 and can be imaged normally, and the area 512 below is affected by the interventional needle 501, and thus, the image quality thereof is reduced.

In an embodiment of the present application, different imaging policies may be used for the difference between the area 511 above and the area 512 below. As shown in FIG. 5c, when imaging is performed by using a two-dimensional probe including a plurality of rows of elements (for the sake of simplifying description, only a first row of elements 521, a second row of elements 522, and a third row of elements 523 of the two-dimensional probe are shown in the example of FIG. 5), the plurality of rows of elements may be respectively used for imaging different areas. At least one row of elements (e.g., the second row of elements 522 shown in FIG. 5) may be used for imaging the area 511 above. Because the area 511 above is not affected by the acoustic impedance of the interventional needle 501, the second row of elements 522 may continue to perform imaging in an original imaging plane to generate a first image (the first image is not shown in FIG. 5). It may be understood that the original imaging plane may include the interventional needle 501, and correspondingly, the second row of elements 522 may image the interventional needle 501. Further, at least one other row of elements (shown in FIG. 5 by taking the first row of elements 521 and the third row of elements 523 as an example) may image at least a part of the area 512 below to generate a second image (the second image is not shown in FIG. 5). As shown in FIG. 5, ultrasound wave transmission directions of the first and third rows of elements 521, 523 and the second row of elements 522 may be located in different planes, so that the first and third rows of elements 521, 523 can “bypass” the interventional needle 501 to image the area below the interventional needle, thereby avoiding interference caused by an acoustic shadow.

Further, images generated by different rows of elements may be synthesized to obtain a synthesized image of a tissue to be imaged. As shown in FIG. 5d, compared with the ultrasound image 500 in FIG. 5a, a synthesized image 502 effectively eliminates an acoustic shadow area below the interventional needle 501, and thus has higher image quality.

An example description of an image synthesis manner is provided below. After the first image is generated by using the at least one row of elements (such as the second row of elements 522) and the first image is generated by using the at least one other row of elements (such as the first and third rows of elements 521, 523), respectively, the following may be performed:

selecting a first portion of the first image that includes the high-acoustic-impedance object and an area above the high-acoustic-impedance object; selecting a second portion of the second image that includes the area below the high-acoustic-impedance object; and synthesizing the first portion and the second portion.

It may be understood that, in the above manner, an acoustic shadow area in the first image can be covered by the second part of the second image, thereby obtaining a synthesized high-quality image.

In the above manner of the present application, ultrasound wave transmission angles of different rows of elements are adjusted, so that the two-dimensional probe performs imaging and synthesis in different imaging planes. Other ultrasound imaging parameters, such as the frequency and power of ultrasound waves of different rows of elements, can be flexibly selected. In some examples, same or similar parameters, e.g., the same frequency, other than the transmission angle may be selected for different rows of elements. In this way, the similarity between the first and second images can be ensured as much as possible, so that the synthesized image is uniform as a whole.

An example description of controlling ultrasound wave transmission directions of different rows of elements to perform ultrasound imaging is provided below.

In some embodiments, performing ultrasound imaging on the high-acoustic-impedance object (e.g., the interventional needle 501) by using at least one row of elements (e.g., the second row of elements 522 in FIG. 5) among the plurality of rows of elements may include:

adjusting, based on relative positions of the high-acoustic-impedance object and the at least one row of elements, an ultrasound wave transmission direction of the at least one row of elements to be perpendicular to a surface of the high-acoustic-impedance object.

In some examples, the ultrasound wave transmission method of the at least one row of elements is adjusted in one imaging plane, and the imaging plane passes through the high-acoustic-impedance object.

FIG. 5 is still used as an example for description. While the second row of elements 522 continues to perform ultrasound imaging in the original imaging plane, relative positions of the second row of elements 522 and the interventional needle 501 may be considered. Specifically, an angle of the second row of elements 522 relative to the interventional needle 501 in the same plane may be considered. Moreover, the ultrasound wave transmission direction of the second row of elements 522 is adjusted based on the angle, to be deflected in the original imaging plane and finally perpendicular to the interventional needle 501. In this way, an ultrasound echo signal of the interventional needle 501 can be enhanced as much as possible without changing the imaging plane, thereby improving the quality of an ultrasound image of the interventional needle 501.

In some other examples, performing ultrasound imaging on the at least a part of the area below by using the at least one other row of elements (e.g., the first and third rows of elements 521, 523 in the example of FIG. 5) may include:

adjusting, based on relative positions of the high-acoustic-impedance object and the at least one other row of elements, an ultrasound wave transmission direction of the at least one other row of elements to at least partially pass through the tissue to be imaged below the high-acoustic-impedance object.

Optionally, the ultrasound wave transmission direction of the at least one other row of elements is adjusted in an imaging plane different from that of the at least one row of elements.

An example description is provided with reference to FIG. 5. In FIG. 5, transmission directions of the first row of elements 521 and the third row of elements 523 may be deflected to avoid the high-acoustic-impedance object (the interventional needle 501) to at least partially pass through the tissue to be imaged in the area 512 below the interventional needle 501. The adjustment of the ultrasound signal transmission directions of the first row of elements 521 and the third row of elements 523 may be adjustment in a different transmission plane. To be specific, the transmission plane may be adjusted from a transmission plane originally substantially the same as that of the second row of elements 522 to a plane intersecting with that of the second row of elements 522, and the planes intersect in the area below the interventional needle 501, that is, the acoustic shadow area. Thus, the first row of elements 521 and the third row of elements 523 are enabled to eliminate the acoustic shadow area.

It may be understood that, in the example corresponding to FIG. 5, three rows of elements are used as an example for exemplary description, but the examples of the present application are not limited thereto. In other examples, the quantity of rows of elements of the two-dimensional probe may alternatively be other numbers, e.g., five rows or seven rows. In addition, in the example of FIG. 5, the middle row of elements is used to continuously image the high-acoustic-impedance object such as the interventional needle, and the elements on both sides are selected to image the acoustic shadow area. However, functions performed by different rows of elements may be adjusted under the teachings of the present disclosure.

In some examples of the present application, the imaging method may be further improved based on a specific use scenario. For example, when the ultrasound imaging method in the present application is used to perform auxiliary imaging in an interventional procedure, the high-acoustic-impedance object may include an interventional object, and correspondingly, the method in the present application may further include:

adjusting ultrasound wave transmission directions of the at least one row of elements and the at least one other row of elements in real time in response to a change in the position of the interventional needle in the tissue to be imaged, to continuously obtain an image of the interventional needle and an image of at least a part of an area below the interventional needle.

In other words, in the case that the position of the high-acoustic-impedance object, that is, the interventional needle, inside the tissue to be imaged may change, the above examples of the present application can also specifically adjust, based on a specific position of the interventional needle, ultrasound waves of the elements for performing imaging. For example, as the interventional needle advances, the ultrasound wave transmission direction of the at least one other row of elements may be adjusted based on a position change of an acoustic shadow area (that is, the area below the interventional needle), thereby ensuring that a high-quality image of the at least a part of the area the interventional needle can be obtained even if the acoustic shadow area changes continuously. Additionally/alternatively, the ultrasound wave transmission direction of the at least one row of elements is also adjusted in real time based on the position of the interventional needle, for example, may be adjusted to be perpendicular to the interventional needle in real time as the position of the interventional needle changes, thereby obtaining a high-quality ultrasound image of the interventional needle.

The inventors have further realized that the imaging efficiency of the elements of the two-dimensional probe may be limited. In view of at least this, some examples of the present application further propose improvements to enable limited elements to be used for imaging of a key anatomical feature as much as possible.

In some examples, the method in the present application further includes: determining a key anatomical feature below the high-acoustic-impedance object based on the identification result, where the key anatomical feature includes at least one of a nodule, a gland, a nerve node, and a blood vessel.

In such an embodiment, the position of the key anatomical feature can be located, thereby providing guidance for subsequent ultrasound imaging.

Further, in a related embodiment, performing ultrasound imaging on the at least a part of the area below by using the at least one other row of elements includes: controlling, based on the position of the structure of interest, an ultrasound wave transmission direction of the at least one other row of elements to at least partially pass through the structure of interest.

In such an embodiment, the at least one other row of elements can be utilized as much as possible for imaging the key anatomical feature. This may be crucial for some use scenarios, such as an interventional procedure. A doctor is often concerned with the key anatomical feature below the interventional needle during the interventional procedure, ensuring that the key anatomical feature is kept a sufficient distance from the interventional needle to avoid damage. The above manner of the present application can preferentially provide acoustic shadow elimination for the key anatomical feature, thereby improving the diagnostic and treatment confidence of the doctor.

In some examples, the method in the present application further includes: displaying the synthesized image in real time; and highlighting the key anatomical feature in the synthesized image.

In this way, it is easier for a doctor to observe the key anatomical feature, thereby avoiding adverse effects such as reduced diagnostic confidence under intervention.

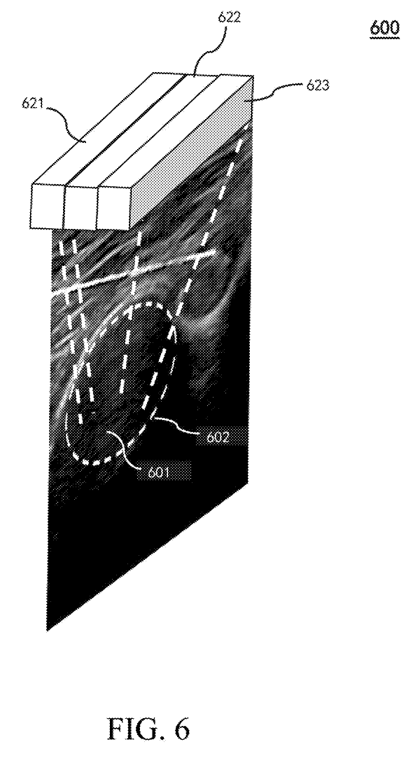

The above embodiment is described below in more detail by using FIG. 6. FIG. 6 is a schematic diagram of imaging a key anatomical feature according to some examples of the present application.

As shown in FIG. 6, an ultrasound image 600 includes a key anatomical feature 601. In the example of FIG. 6, the key anatomical feature 601 is shown in a form of a nodule (e.g., a greater tuberosity of the humerus, GT). In this example, image identification is not only used to identify a high-acoustic-impedance object such as an interventional needle, but also includes identification of the key anatomical feature 601, to determine detailed information such as the size and position of the key anatomical feature.

Further, at least one row of elements, such as a first row of elements 621 and a third row of elements 623, may be controlled to transmit and receive ultrasound waves in a targeted manner toward the key anatomical feature 601, so that as many limited ultrasound signal paths as possible can be allocated to the key anatomical feature 601. For a specific manner of controlling the elements, refer to that described in any one of the above examples of the present application. The manner includes a manner of controlling a second row of elements 622, and details are not described herein again.

It may be understood that the above process of identifying the key anatomical feature 601 may be prior to an interventional procedure intervention, for example, when an interventional needle has not yet entered the tissue to be imaged. Alternatively, the process may be during the very course of the interventional procedure, for example, when the interventional needle has just entered the tissue to be imaged. The present application does not make a unique definition.

The ultrasound image 600 shown in FIG. 6 is a synthesized image obtained by the image synthesis method in the present application. In some examples, in addition to displaying the synthesized image in real time, the identified key anatomical feature 601 may also be highlighted. The highlighting may be performed in such a way that an outer contour 602 of the key anatomical feature 601 is displayed as shown in FIG. 6. Thus, a doctor can more easily observe a specific position of the key anatomical feature 601 when observing the image, thereby making a timely and accurate clinical determination.

So far, a plurality of ultrasound imaging methods and the effects thereof have been described above in the present application. In another example, the present disclosure further provides an ultrasound imaging system, including:
a two-dimensional probe; and
a processor. The processor is configured to perform the method described in any one of the above examples of the present application.

It may be understood that, for a specific structure of the two-dimensional probe, reference may be made to that described in any one of the above examples of the present application or in the prior art. Similarly, for the processor, reference may be made to that described in FIG. 1 or another example of the present application. For example, the processor may be one or more processors in the controller circuit 102 in FIG. 1. In addition, the ultrasound imaging system may further include another component described in any one of the above examples of the present application, and details are not described herein again.

An embodiment of the present disclosure further provides a computer program product, including computer-readable codes or a non-transitory computer-readable storage medium loaded with computer-readable codes, where when the computer-readable codes are run in a processor of a medical device, the processor in the medical device performs the steps of the foregoing method.

Some examples of the present disclosure further provide a non-transitory computer-readable medium. The non-transitory computer-readable medium stores a computer program, the computer program has at least one code segment, and the at least one code segment is executable by a machine to cause the machine to perform the steps of the method described in any one of the above examples of the present application.

The computer-readable storage medium may be a tangible device that can hold and store instructions used by an instruction execution device. The computer-readable storage medium may be, for example, but is not limited, to an electrical storage device, a magnetic storage device, an optical storage device, an electromagnetic storage device, a semiconductor memory device, or any suitable combination thereof. More specific examples (a non-exhaustive list) of the computer-readable storage medium include: a portable computer disk, a hard disk, a random access memory (RAM), a read only memory (ROM), an erasable programmable read only memory (EPROM or flash memory), a static random access memory (SRAM), a portable compact disc read-only memory (CD ROM), a digital versatile disk (DVD), a memory stick, a floppy disk, mechanical coding equipment, such as a punch card having instructions stored thereon or a structure of bumps within recessions, and any suitable combination thereof. The computer-readable storage medium used herein is not interpreted as transient signals themselves, such as radio waves or other freely propagated electromagnetic waves, electromagnetic waves propagated through a waveguide or other transmission media (e.g., light pulses passing through a fiber optic cable), or electrical signals transmitted through electric wires.

The computer-readable program instructions described herein may be downloaded from a computer-readable storage medium to various computing/processing devices or downloaded to an external computer or external storage device via a network such as the Internet, a local area network, a wide area network and/or a wireless network. The network may include copper transmission cables, fiber transmission, wireless transmission, routers, firewalls, switches, gateway computers, and/or edge servers. A network adapter card or a network interface in each computing/processing device receives computer-readable program instructions from the network and forwards the computer-readable program instructions, for storing the same in a computer-readable storage medium in each computing/processing device.

Computer program instructions for executing the operations of the present disclosure can be assembly instructions, instruction set architecture (ISA) instructions, machine instructions, machine-related instructions, microcode, firmware instructions, state setting data, or source code or object code written in any combination of one or more programming languages, the programming language including object oriented programming languages such as Smalltalk, C++, and the like, and conventional procedural programming languages such as the “C” language or similar programming languages. The computer-readable program instructions can be executed entirely or partly on a user computer, executed as a stand-alone software package, executed partly on a user computer and partly on a remote computer, or executed entirely on a remote computer or server. In the case of a remote computer, the remote computer may be connected to a user computer through any kind of network, including a local area network (LAN) or a wide area network (WAN), or connected to an external computer (for example, through the Internet, using an Internet service provider). In some embodiments, an electronic circuit, for example, a programmable logic circuit, a field-programmable gate array (FPGA), or a programmable logic array (PLA), may execute the computer-readable program instructions by utilizing state information of the computer-readable program instructions to personalize the electronic circuit, in order to implement various aspects of the present disclosure.

The purpose of providing the above specific embodiments is to allow the disclosure of the present application to be understood more thoroughly and comprehensively; however, the present application is not limited to these specific embodiments. A person skilled in the art should understand that various modifications, equivalent replacements, changes, and the like can be further made to the present application and should be included in the scope of protection of the present application as long as these changes do not depart from the spirit of the present application.

What is claimed is:

1. A method for performing ultrasound imaging with a two-dimensional probe, wherein the two-dimensional probe comprises a plurality of rows of elements, the method comprising:
obtaining a two-dimensional ultrasound image of a tissue to be imaged;
performing identification on the ultrasound image, and determining a high-acoustic-impedance object in the ultrasound image based on an identification result;
continuing to perform ultrasound imaging on the high-acoustic-impedance object by using at least one row of elements among the plurality of rows of elements to obtain a first image of the high-acoustic-impedance object;
performing ultrasound imaging on at least a part of an area below the high-acoustic-impedance object by using at least one other row of elements among the plurality of rows of elements to obtain a second image of the at least a part of the area below; and
synthesizing the first image and the second image to obtain a real-time synthesized image.

2. The method according to claim 1, wherein the high-acoustic-impedance object comprises at least one of an interventional needle, a bone, a stone, and a calcification.

3. The method according to claim 1, wherein continuing to perform ultrasound imaging on the high-acoustic-impedance object by using at least one row of elements among the plurality of rows of elements comprises:
adjusting, based on relative positions of the high-acoustic-impedance object and the at least one row of elements, an ultrasound wave transmission direction of the at least one row of elements to be perpendicular to a surface of the high-acoustic-impedance object.

4. The method according to claim 3, wherein the ultrasound wave transmission method of the at least one row of elements is adjusted in one imaging plane, and the imaging plane passes through the high-acoustic-impedance object.

5. The method according to claim 1, wherein performing ultrasound imaging on the at least a part of the area below by using the at least one other row of elements comprises:
adjusting, based on relative positions of the high-acoustic-impedance object and the at least one other row of elements, an ultrasound wave transmission direction of the at least one other row of elements to at least partially pass through the tissue to be imaged below the high-acoustic-impedance object.

6. The method according to claim 5, wherein the ultrasound wave transmission direction of the at least one other row of elements is adjusted in an imaging plane different from that of the at least one row of elements.

7. The method according to claim 1, further comprising:
determining a key anatomical feature below the high-acoustic-impedance object based on the identification result, wherein the key anatomical feature comprises at least one of a nodule, a gland, a nerve node, and a blood vessel.

8. The method according to claim 7, wherein performing ultrasound imaging on the at least a part of the area below by using the at least one other row of elements comprises:
controlling, based on a position of the key anatomical feature, an ultrasound wave transmission direction of the at least one other row of elements to at least partially pass through the key anatomical feature.

9. The method according to claim 8, further comprising:
displaying the synthesized image in real time; and
highlighting the key anatomical feature in the synthesized image.

10. The method according to claim 1, wherein the plurality of rows of elements comprises three rows of elements, the at least one row of elements comprises a middle row of elements among the three rows of elements, and the at least one other row of elements comprises elements on both sides among the three rows of elements.

11. The method according to claim 1, wherein synthesizing the first image and the second image comprises:
selecting a first portion of the first image that comprises the high-acoustic-impedance object and an area above the high-acoustic-impedance object;
selecting a second portion of the second image that comprises the area below the high-acoustic-impedance object; and
synthesizing the first portion and the second portion.

12. The method according to claim 1, wherein the high-acoustic-impedance object comprises an interventional needle, and the method further comprises:
adjusting ultrasound wave transmission directions of the at least one row of elements and the at least one other row of elements in real time in response to a change in the position of the interventional needle in the tissue to be imaged, to continuously obtain an image of the interventional needle and an image of at least a part of an area below the interventional needle.

13. An ultrasound imaging system, comprising:
a two-dimensional probe including a plurality of rows of transducer elements;
a memory storing instructions; and
a processor configured to execute the instructions to:
obtain a two-dimensional ultrasound image of a tissue to be imaged;
perform identification on the ultrasound image, and determining a high-acoustic-impedance object in the ultrasound image based on an identification result;
continue to perform ultrasound imaging on the high-acoustic-impedance object by using at least one row of elements among the plurality of rows of elements to obtain a first image of the high-acoustic-impedance object;
perform ultrasound imaging on at least a part of an area below the high-acoustic-impedance object by using at least one other row of elements among the plurality of rows of elements to obtain a second image of the at least a part of the area below; and
synthesize the first image and the second image to obtain a real-time synthesized image.

14. A non-transitory computer-readable medium storing instruction thereon that, when executed by a computer, cause the computer to:
obtain a two-dimensional ultrasound image of a tissue to be imaged using a two-dimensional probe including a plurality of rows of transducer elements;
perform identification on the ultrasound image, and determining a high-acoustic-impedance object in the ultrasound image based on an identification result;
continue to perform ultrasound imaging on the high-acoustic-impedance object by using at least one row of elements among the plurality of rows of elements to obtain a first image of the high-acoustic-impedance object;
perform ultrasound imaging on at least a part of an area below the high-acoustic-impedance object by using at least one other row of elements among the plurality of rows of elements to obtain a second image of the at least a part of the area below; and
synthesizing the first image and the second image to obtain a real-time synthesized image.
